(19) United States
(12) Patent Application Publication
(10) Pub. No.: US 2017/0225032 A1
(43) Pub. Date: Aug. 10, 2017

(54) EXERCISE TRAINING SYSTEM

(71) Applicant: Darnell Jones, Irving, TX (US)

(72) Inventor: Darnell Jones, Irving, TX (US)

(21) Appl. No.: 15/427,558

(22) Filed: Feb. 8, 2017

Related U.S. Application Data
(60) Provisional application No. 62/292,997, filed on Feb. 9, 2016.

(51) Int. Cl. A63B 24/00; A63B 21/065; A43B 5/00; A43B 3/00; A63B 71/12; A63B 71/10; A41D 20/00; A42B 3/04; A63B 21/00; A63B 69/00; A63B 69/20; A63B 69/38; A63B 71/06; A61B 5/021; A61B 5/00; A61B 5/01; A61B 5/145; A41D 1/00

(52) U.S. Cl. CPC A63B 24/0006; A41D 1/002; A63B 21/065; A43B 5/00; A43B 3/0005; A63B 71/12; A63B 71/1225; A63B 71/10; A41D 20/00; A42B 3/0433; A63B 21/4009; A63B 21/4015; A63B 21/4019; A63B 21/4021; A63B 21/4005; A63B 21/4003; A63B 21/4011; A63B 21/4017; A63B 69/00; A63B 69/0071; A63B 69/004; A63B 69/0002; A63B 69/20; A63B 69/38; A63B 69/0095; A63B 69/002; A63B 71/0622; A63B 24/0062; A61B 5/021; A61B 5/0059; A61B 5/01; A61B 5/14542; A61B 5/6806; A61B 5/6803; A61B 5/6807; A63B 2220/40; A63B 2220/56; A63B 2220/803; A63B 2230/50; A63B 2230/30; A63B 2230/06; A63B 2230/207; A63B 2071/125; A63B 2071/1258; A41D 2600/10; A63B 2209/00; A63B 2220/836; A63B 2225/50; A63B 2220/805; A61B 2562/0219; A61B 2562/0247

(57) ABSTRACT

A training system, kit, and method including a weighted wearable equipment, e.g. gloves, having a sensor (e.g. accelerometers, gyroscopes, photoelectric sensors, position sensors, tilt sensors, pressure sensors, temperature sensors, blood pressure sensors, heart rate monitors, and SpO2 sensors) and including a weight enhancement (e.g. weight bodies in closed pockets); a non-weighted wearable equipment of the same type as the weighted wearable equipment, the non-weighted wearable equipment having a sensor and. not including a weight enhancement; and a training application in functional communication with each of the weighted wearable equipment and the non-weighted wearable equipment and having a data processor that includes instructions for: analyzing data received from the sensor of each of the weighted wearable equipment and the non-weighted wearable equipment and generating predictive information derived from exercise training data from the sensors.

### CROSS-REFERENCE TO RELATED APPLICATIONS

This invention claims priority, under 35 U.S.C. §120, to the U.S. Provisional Patent Application No. 62/292,997 to Darnell Jones filed on 9 Feb., 2016, which is incorporated by reference herein in its entirety.

### BACKGROUND OF THE INVENTION

Field of the Invention

The present invention relates to training devices, specifically an exercise training system with physical attribute sensors.

Description of the Related Art

Exercise and sports training activities have been an important human endeavor for thousands of years. Various devices and techniques have been developed to facilitate training, to make it more efficient/effective, and/or to allow for training during times and in places where it would not otherwise be possible.

In furtherance of this, exercise devises, like treadmills, stationary bicycles, home gym equipment and the like have been developed. These devices simulate conditions and/or situations where muscles may be repetitively used in a coordinated manner. Thus, users may walk, run, push, pull, row, climb, lift, or otherwise repetitively move in a manner that allows for muscle growth and/or improvements in coordination.

Advances in technology have allowed for computers and sensors that are able to observe and record physical characteristics. Accordingly, there are devices and systems that incorporate more modem technology into exercise systems. There are stationary bicycles and treadmills that track and record motion, that automatically alter resistance according to programed sequences, and that detect heart rate and map the same against an exercise program.

Further uses of advanced electronics/computing and/or exercise technology have been developed. Some improvements have been made in the field. Examples of references related to the present invention are described below in their own words, and the supporting teachings of each reference are incorporated by reference herein:

U.S. Pat. No. 5,184,319 which teaches a man-machine interface which provides force and texture information to sensing body parts. The interface is comprised of a force actuating device that produces a force which is transmitted to a force applying device. The force applying device applies the generated force to a pressure sensing body part. A force sensor on the force applying device measures the actual force applied to the pressure sensing body part, while angle sensors measure the angles of relevant joint body parts. A computing device uses the joint body part position information to determine a desired force value to be applied to the pressure sensing body part. The computing device combines the joint body part position information with the force sensor information to calculate the force command which is sent to the force actuating device. In this manner, the computing device may control the actual force applied to a pressure sensing body part to a desired force which depends upon the positions of related joint body parts. In addition, the interface is comprised of a displacement actuating device which produces a displacement which is transmitted to a displacement applying device (e.g., a texture simulator). The displacement applying device applies the generated displacement to a pressure sensing body part. The force applying device and displacement applying device may be combined to simultaneously provide force and displacement information to a pressure sensing body part.

U.S. Pat. No. 6,157,898 teaches a device for measuring a movable object, such as a baseball, football, hockey puck, soccer ball, tennis ball, bowling ball, or a golf Part of the device, called the object unit, is embedded, secured, or attached to the movable object of interest, and consists of an accelerometer network, electronic processor circuit, and a radio transmitter. The other part of the device, called the monitor unit, is held or worn by the user and serves as the user interface for the device. The monitor unit has a radio receiver, a processor, an input keypad, and an output display that shows the various measured motion characteristics of the movable object, such as the distance, time of flight, speed, trajectory height, spin rate, or curve of the movable object, and allows the user to input data to the device.

U.S. Pat. No. 6,640,202 teaches an apparatus, method, and system for determining the shape of a three dimensional object. In a preferred embodiment, the apparatus includes an array of sensors and elastic connections between the sensors within the array. When placed over a three dimensional object, the array of sensors deforms to conform to the surface topology of the three dimensional object. The sensors are connected to a data processor in which the data from the sensors is taken to construct a three-dimensional representation of the actual physical three dimensional object; and

U.S. Patent Publication No.: 2014/0295757 teaches improving the usability of an electronic device, the electronic device includes: a first communication unit located near a user; a receive unit configured to receive data through communication via a body of the user or near field communication between a second communication unit located in a member used by the user and the first communication unit; and a recording unit configured to record data pertaining to the member when communication is established between the first communication unit and the second communication unit;

U.S. Patent Publication No.: 2006/0071912 teaches a touch sensitive input device that uses vibrations due to touch impacts and/or frictional movement of a touch implement across a surface to determine information related to the touch, such as touch position. The present invention also provides for detecting lift-off events in such vibration sensing input devices. Lift-off detection can be accomplished by monitoring for a signal that indicates a sustained touch on the touch plate, and correlating a change in such a signal with a lift-off event. Signals indicating a sustained touch can include low frequency rumbles coupled into the h plate via the touch implement, touch plate bending under the force of a sustained touch, and touch plate displacement under the force of a sustained touch;

U.S. Patent Publication No.: 2011/0302694 teaches a clinical sensing glove system to quantify force, shear, hardness, etc., measured in manual therapies is disclosed. A sensor is disposed in a clinical glove. The sensor undergoes micro-bending, macro-bending, evanescent coupling, a change in resonance, a change in polarization, a change in phase modulation, in response to pressure/force applied. The amount of micro-bending, macro-bending, evanescent coupling, change in resonance, change in polarization, and/or change in phase modulation is proportional to the intensity of the pressure/force. A clinician can quantitatively determine the amount of pressure, force, shear, hardness, rotation, etc., applied;

U.S. Patent Publication No.: 2013/0060166 teaches a method, system, and/or device for providing rehabilitation and assessing function from a portion of the human body. In one embodiment, there is disclosed a method, system, and/or device for providing rehabilitation and assessing of hand function using an audio interface. The audio interface may be a music-based interface and device may include a monitor unit, such as a hand monitoring unit, for providing data of a movement to a computing device, such as a microcontroller. The computing device may output data to a music-based interface;

U.S. Patent Publication No.: 2013/0158365 teaches a probe system that includes a finger-mountable housing having a distal end and a proximal receptacle end. The proximal receptacle end defines an opening to receive a finger. The probe system also includes a probe assembly disposed on or within the finger-mountable housing and having at least a first sensor. The first sensor is positioned to measure a physical characteristic of a first tissue when the finger-mountable housing and probe assembly are inserted in a rectum of the patient;

U.S. Patent Publication No.: 2013/0197399 teaches a patient evaluation apparatus includes a glove body adapted to be worn on an examiner's hand, finger orientation sensors mounted to the glove body adapted to sense the orientation of the fingers and thumb of the examiner's hand, three sensors mounted to the glove body adapted to measure threes applied against the examiner's hand, and a motion sensor mounted to the glove body adapted to detect motion of the examiner's hand; and

U.S. Patent Publication No.: 2014/0364771 teaches in regards to pressure sensitive devices, systems and methods for alerting a user of movements potentially adverse to health or surgical recovery are disclosed. The pressure sensitive device may include a force sensor placed along the anterior aspect of a hand; and a vibration motor in communication with the force sensor in close proximity to the user, e.g., along the posterior aspect of the wrist. The vibration motor is configured to vibrate upon the measured force exceeding a predetermined threshold. This threshold can be adjusted according to clinical application and/or user need. The pressure sensitive device may further include a memory chip or wireless transmitter for recording and relaying data associated with a patient profile, and is enabled to interface with a sensing technology. Logged data may be used for patient rehabilitation.

The inventions heretofore known suffer from a number of disadvantages, which may include one or more of failing to show training progress and/or improvement; providing poor training; failing to provide training data or sufficient training data; failing to show progress in specific areas and/or techniques; failing to provide predictive information during training; not providing enough information to trainers to allow them to improve training protocols; and/or failing to allow for better choices with regard to specific drills to perform and/or their durations.

What is needed is an exercise training system/device that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available sports-training ball assembly. Accordingly, the present invention has been developed to provide an exercise training system, device and method.

In one nonlimiting embodiment, there is a training system that may be over a computerized network. The system may include one or more of the following: a weighted wearable equipment that may be of a type having a sensor and that may include a weight enhancement; a non-weighted wearable equipment that may be of the same type as the weighted wearable equipment, the non-weighted wearable equipment may have a sensor and/or may not include a weight enhancement; and/or a training application that may be in functional communication with one or more of the weighted wearable equipment and/or the non-weighted wearable equipment and that may have a data processor that may include instructions for one or more of: analyzing data received from the sensor of one or more of the weighted wearable equipment and the non-weighted wearable equipment and/or generating predictive information derived from exercise training data from one or more of the sensors.

The wearable equipment of the training system may be gloves, shoes, belts, shoulder-pads, knee-pads, elbow-pads, helmets, wristbands, and/or shin-guards. The sensor(s) may be accelerometers, gyroscopes, photoelectric sensors, position sensors, tilt sensors, pressure sensors, temperature sensors, blood pressure sensors, heart rate monitors, and/or SpO2 sensors. The weight enhancement may include a plurality of weight bodies disposed in closed pockets within the weighted wearable equipment.

The instructions of the data processor may include instructions for generating predictive information about how a user will currently perform using the non-weighted wearable equipment and such may be based on generating a mapping rule , such as but not limited to, by comparing historical data for that user from both the weighted wearable equipment sensor and the non-weighted wearable equipment sensor and/or may be by applying a mapping rule to current sensor data from the weighted wearable equipment sensor. The data processor may receive motion information from the sensors. There may be an analysis module that may include one or more of: a data processor, a data storage module that may be functionally coupled to the analysis module such that the analysis module may call data therefrom, and/or a user interface module that may be functionally coupled to the data processor module such that analysis therefrom may be reported to the user interface module on demand from a user.

In another non-limiting embodiment, there is a a training system that includes onr or more of: a weighted glove that may have an accelerometer and/or may include a weight enhancement; a non-weighted glove that may have an accelermeter and/or may not include a weight enhancement; and/or a training application that may be in functional communication with each of the weighted glove and the non-weighted glove and/or may have a data processor that may include instructions for one or more of analyzing exercise training data received from the accelerometer of each of the weighted wearable equipment and the non-weighted wearable equipment; and/or generating predictive performance data derived from analyzing the exercise training data.

It may be that instructions for generating predictive performance data include instructions for generating a mapping rule by comparing historical data for that user from both the weighted glove and the non-weighted glove and by applying the mapping rule to current accelerometer data from the weighted glove.

There may be a user interface module that may be disposed on a portable computing device that may be in functional communication with the data processor such that a user of the portable computing device can receive predictive performance data therefrom. The user interface module may include a user interface for an athlete account that may be different from a user interface for a coach account. It may be that each of the athlete account and the coach account can access the same set of training and predictive data over different mobile computing devices.

It may be that each of the weighted and non-weighted gloves includes a wireless communication module that may transmit training data to a mobile computing device.

It may be that one or more of the weighted glove and non-weighted glove includes a plurality of sensor types.

In still another non-limiting embodiment, there is a training system for use in weight-enhanced training techniques, that may include one or more of: a first sensor module that may be disposed within a first apparel; a second sensor module that may be disposed within a second apparel, wherein the second apparel may be of a same type as the first apparel and/or may have a weight differential with respect to the first apparel; an analysis module that may be in functional communication with one or more of the first sensor module and the second sensor module, wherein the analysis module may include instructions for receiving and/or processing sensor information from one or more of the first sensor module and the second sensor module and/or associating such data with one or more respectively and/or wherein the analysis module may include information about the weight differential and/or utilizes that information in processing the sensor information.

It may be that each of the first apparel and second apparel are gloves that may include an accelerometer within one or more of the associated first and second sensor modules. There may be a predictive module that may be functionally coupled to the analysis module and/or include instructions for predicting performance of a user that may be based on historical sensor data.

In still yet another non-limiting embodiment, there may be a training kit, that may include one or more of: a weighted wearable equipment that may be of a type having a sensor and/or may include a weight enhancement; a non-weighted wearable equipment that may be of the same type as the weighted wearable sensor, wherein the non-weighted wearable equipment may have a sensor and/or may not include a weight enhancement; and/or instructions for accessing a training application that may he able to analyze data received from the sensor of one or more of the weighted wearable equipment and/or the non-weighted wearable equipment and/or may generate predictive information that may be derived from exercise training data from the sensors.

It may be that the type is a glove and/or the sensor of one or more of the gloves is an accelerometer.

In still yet another further embodiment, there may be a method of training, comprising one or more of the steps of: collecting weighted training data for a user from a weighted wearable equipment that may be of a type having a sensor and/or including a weight enhancement; collecting non-weighted training data for the user that may be from a non-weighted wearable equipment that may be of the same type as the weighted wearable sensor, and/or wherein the non-weighted wearable equipment may have a sensor and/or not include a weight enhancement; analyzing weighted and/or non-weighted training data that may be for the user that may be in combination with information about a weight differential between the weighted wearable equipment and the non-weighted wearable equipment; and/or generating predictive performance data that may be for the user that may be derived from analyzing the weighted and/or non-weighted training data.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a network diagram of an exercise training system, according to one embodiment of the invention;

FIG. 2 is a module diagram of an equipment, according to one embodiment of the invention;

FIG. 3 is a module diagram of an application module, according to one embodiment of the invention;

FIG. 4 is a module diagram of a backend services module, according to one embodiment of the invention;

FIG. 5 is a module diagram of a training kit, according to one embodiment of the invention;

FIG. 6 is a flowchart of a method of training according to one embodiment of the invention.

FIG. 7 is a prophetic view of a screen of a smartphone displaying predictive information based on sensor data, according to one embodiment of the invention

FIG. 8 is a top perspective view of a non-weighted training glove with sensors of an exercise training system, according to one embodiment of the invention;

FIG. 9 is a bottom perspective view of a weighted glove with sensors of an exercise training system, according to one embodiment of the invention; and

FIG. 10 is a perspective view of a weighted glove and a non-weighted glove of an exercise training system about to catch a football, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

Many of the functional units described in this specification have been labeled as modules in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like. Modules may also be implemented in software for execution by various types of processors. An identified module of programmable or executable code may, for instance, comprise one or more physical or logical blocks of computer instructions which may, for instance, be organized as an object, procedure, or function.

Nevertheless, the executables of an identified module need not be physically located together, but may comprise disparate instructions stored in different locations which, when joined logically together, comprise the module and achieve the stated purpose for the module. Indeed, a module and/or a program of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

The various system components and/or modules discussed herein may include one or more of the following: a host server, motherboard, network, chipset or other computing system including a processor for processing digital data; a memory device coupled to a processor for storing digital data; an input digitizer coupled to a processor for inputting digital data; an application program stored in a memory device and accessible by a processor for directing processing of digital data by the processor; a display device coupled to a processor and/or a memory device for displaying information derived from digital data processed by the processor; and a plurality of databases including memory device(s) and/or hardware/software driven logical data storage structure(s).

Various databases/memory devices described herein may include records associated with one or more functions, purposes, intended beneficiaries, benefits and the like of one or more modules as described herein or as one of ordinary skill in the art would recognize as appropriate and/or like data useful in the operation of the present invention.

As those skilled in the art will appreciate, any computers discussed herein may include an operating system, such as but not limited to: Android, iOS, BSD, IBM z/OS, Windows Phone, Windows CE, Palm OS, Windows Vista, NT, 95/98/2000, OS X, OS2; QNX, UNIX; GNU/Linux; Solaris; MacOS; and etc., as well as various conventional support software and drivers typically associated with computers. The computers may be in a home, industrial or business environment with access to a network. In an exemplary embodiment, access is through the Internet through a commercially-available web-browser software package, including but not limited to Internet Explorer, Google Chrome, Firefox, Opera, and Safari.

The present invention may be described herein in terms of functional block components, functions, options, screen shots, user interactions, optional selections, various processing steps, features, user interfaces, and the like. Each of such described herein may be one or more modules in exemplary embodiments of the invention even if not expressly named herein as being a module. It should be appreciated that such functional blocks and etc. may be realized by any number of hardware and/or software components configured to perform the specified functions. For example, the present invention may employ various integrated circuit components, e.g., memory elements, processing elements, logic elements, scripts, look-up tables, and the like, which may carry out a variety of functions under the control of one or more microprocessors or other control devices. Similarly, the software elements of the present invention may be implemented with any programming or scripting language such as but not limited to Eiffel, Haskell, C, C++, Java, Python, COBOL, Ruby, assembler, Groovy, PERL, Ada, Visual Basic, SQL Stored Procedures, AJAX, Bean Shell, and extensible markup language (XML), with the various algorithms being implemented with any combination of data structures, objects, processes, routines or other programming elements. Further, it should be noted that the present invention may employ any number of conventional techniques for data transmission, signaling, data processing, network control, and the like. Still further, the invention may detect or prevent security issues with a client-side scripting language, such as JavaScript, VBScript or the like.

Additionally, many of the functional units and/or modules herein are described as being “in communication” with other functional units, third party devices/systems and/or modules. Being “in communication” refers to any manner and/or way in which functional units and/or modules, such as, but not limited to, computers, networks, mobile devices, program blocks, chips, scripts, drivers, instruction sets, databases and other types of hardware and/or software, may be in communication with each other. Some non-limiting examples include communicating, sending, and/or receiving data and metadata via: a wired network, a wireless network, shared access databases, circuitry, phone lines, internet backbones, transponders, network cards, busses, satellite signals, electric signals, electrical and magnetic fields and/or pulses, and/or so forth.

As used herein, the term “network” includes any electronic communications means which incorporates both hardware and software components of such. Communication among the parties in accordance with the present invention may be accomplished through any suitable communication channels, such as, for example, a telephone network, an extranet, an intranet, Internet, point of interaction device (point of sale device, personal digital assistant, cellular phone, kiosk, etc.), online communications, off-line communications, wireless communications, transponder communications, local area network (LAN), wide area network (WAN), networked or linked devices and/or the like. Moreover, although the invention may be implemented with TCP/IP communications protocols, the invention may also be implemented using other protocols, including but not limited to IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997), the contents of which are hereby incorporated by reference.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, for example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

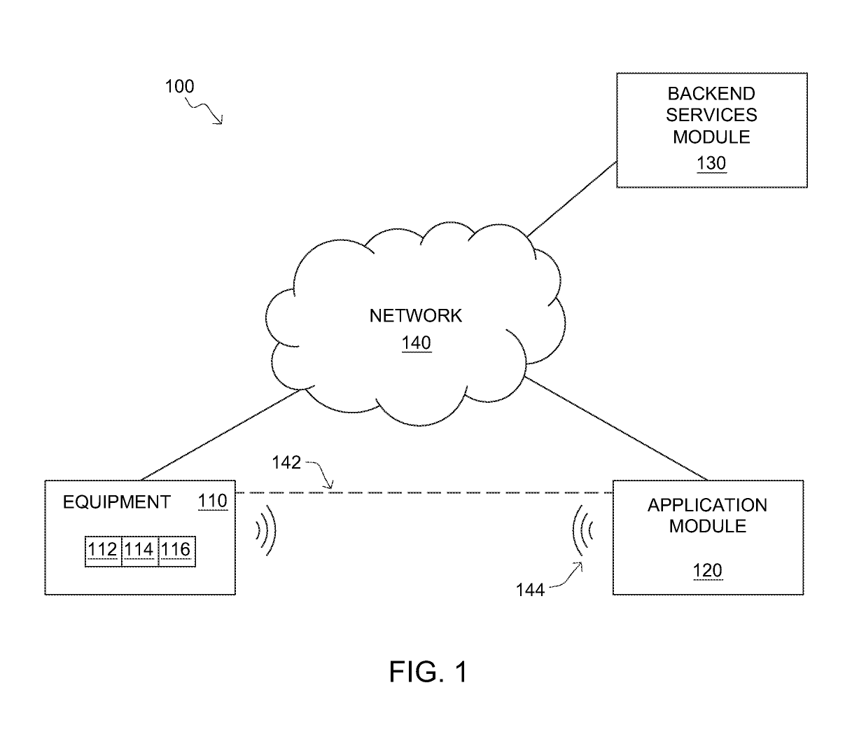

FIG. 1 is a network diagram of an exercise training system, according to one embodiment of the invention. There is shown a network 140 in communication with each of an equipment 110, an application module 120, and a backend services module 130. The illustrated equipment and modules are thereby able to communicate with each other and/or share data/information as appropriate for their integrated functioning.

The illustrated equipment 110 allows and/or facilitates exercise and/or training for one or more users. The illustrated equipment is in communication with the network 140, may have direct communication 142 with the illustrated application module and/or may include wireless communication capabilities. Communication with the various modules/networks described herein may be persistent or may be occasional due to proximity and/or due to instances of connectivity. As a non-limiting example, there may be direct connection between equipment and an application when a cord is coupled between the equipment and a smartphone with an app installed thereto, but no direct communication while the equipment is in use because it is not so coupled. As another example, there may be a memory device that may be exchanged between the equipment and another module/device described herein so that communication between the two is always through the intermediary memory device.

The illustrated equipment 110 includes a plurality of equipment 112, 114, and 116 that a user may use in exercise and/or sports training. Such may include one or more of exercise devices, systems, apparel, gear, tools and the like. There may be a weighted wearable equipment of a type having a sensor and including a weight enhancement; and a non-weighted wearable equipment of the same type as the weighted wearable equipment, the non-weighted wearable equipment having a sensor and not including a weight enhancement.

According to one embodiment of the invention, there is a weighted training glove of an exercise training system. The weighted training glove includes a plurality of weights disposed about a backside of the training glove. The weighted training glove includes an array of four finger regions and each finger region may have at least two weights coupled thereto in a longitudinally spaced relationship to each other. The weighted training glove includes a thumb region that may be spaced and orientated away from the finger region. The thumb region may include a weight that may be coupled thereto.

The weighted training glove may include a weight that may be coupled to the finger region of the weighted training glove. The weight may include a sealed pocket that may have heavy grains. The weight may not be selectably removable. The glove may include a weighted sleeve that may be extending proximally from a proximal end of the combined dorsal and palmar panels.

The illustrated application module 120 allows/facilitates as user's interaction and/or awareness of data from the equipment. The application module provides a user interface for the user in relation to the equipment. The application module may be resident on a portable computing device, such as but not limited to a smartphone, laptop, smart watch, and the like and combinations thereof. Thus, the user is able to view information associated with the equipment.

The illustrated application module 120 includes software/hardware having a user interface to allow a user to view data (and/or resultant analysis) from the equipment. There may be a training application in functional communication with each of the weighted wearable equipment and the non-weighted wearable equipment and having a data processor that includes instructions for one or more of: analyzing data received from the sensor of each of the weighted wearable equipment and the non-weighted wearable equipment and generating predictive information derived from exercise training data from the sensors.

The illustrated backend services module 130 provides overarching management and control for the training system. Such may include but is not limited to providing: updates, account management, communication of information between accounts, managing account permissions, distributing new analysis protocols, aggregating training data, anonymizing training data, improving analysis algorithms, and the like and combinations thereof. The backend services module 130 may also authorize access to tools/resources used within the system, provide configuration information for equipment, and/or integrate various equipment into the system.

The illustrated network 140 provides communication between the various components described herein. Such may be over an internet/intranet and/or over various memory devices and data transmission systems, Such may be a persistent network or may exist on occasions where connectivity is established, but otherwise not.

In one nonlimiting embodiment, there is a backend services module that is accessible by the equipment and/or the application module over an internet/intranet network, such as by operation of a web page over the internet and/or a server to which an application has a connection. The application module and the equipment may each include a memory storage device port that uses a compatible memory storage device that may be used with the equipment while training and thereby gathering data and then moved to the application module for data retrieval. In another non-limiting embodiment, the equipment is in wireless (e.g. Bluetooth) communication with the application module which is coupled to the backend services module over the Internet. Thereby the equipment is indirectly coupled to the backend services module through the application module.

In one non-limiting embodiment, there is a training system, including a weighted glove having an accelerometer and including a weight enhancement; a non-weighted glove having an accelerometer and not including a weight enhancement; and a training application in functional communication with each of the weighted glove and the non-weighted glove and having a data processor that includes instructions for: analyzing exercise training data received from the accelerometer of each of the weighted wearable equipment and the non-weighted wearable equipment and/or generating predictive performance data derived from analyzing the exercise training data.

In another non-limiting embodiment, there is a training system for use in weight-enhanced training techniques, comprising: a first sensor module disposed within a first apparel; a second sensor module disposed within a second apparel, the second apparel being of a same type as the first apparel but having a weight differential with respect to the first apparel; an analysis module in functional communication with each of the first sensor module and the second sensor module, the analysis module including instructions for receiving and processing sensor information from each of the first sensor module and the second sensor module and associating such data with each respectively and wherein the analysis module includes information about the t differential and utilizes that information in processing the sensor information. The apparel may include, but is not limited to: gloves, shoes, socks, boots, sports/professional body armor, helmets, body pads, shirts, shorts, pants, belts, body-part braces, and the like and combinations thereof.

The training system may include where each of the first apparel and second apparel are gloves that each include an accelerometer within the associated first and second sensor modules. There may also be a predictive module that may be functionally coupled to the analysis module and/or includes instructions for predicting performance of a user based on historical sensor data.

According to one embodiment of the invention, there is an exercise training system including a plurality of pairs of training gloves and/or other clothing/equipment items, such as but not limited to fingerless gloves, padded, fight gloves, hand-wraps, baseball mitts, climbing gloves, work gloves, construction gloves, winter gloves, mittens, safety gloves, biker gloves, gauntlets, golf gloves, chainsaw gloves, shoes, boots, vests, helmets, pads (e.g. shoulder, knee), and the like and/or combinations thereof. The gloves/equipment comes in sets, with at least one being weighted (having bodies of mass that serve to increase the weight such as but not limited to pouches of shot/beads/sand/pellets/powders/gels/fluids of heavy materials such as but not limited to lead, steel, water, iron, ceramic, plastics, clay, sand, foam and the like and combinations thereof) and one non-weighted (similar in all respects to the weighted version, but not including the weight(s)). There may be a multiplicity of gloves/equipment each having different amounts of weights. At least the weighted and non-weighted training gloves/equipment are each in communication (selectably or otherwise) with an exercise training app or a software application via one or more sensors that measure one or more characteristics, including but not limited to speed, acceleration, pressure, angle, orientation, impact, velocity, movement, position and the like and combinations thereof. Such sensors may measure/observe such characteristics directly and/or may determine such through calculation or other algorithm(s) either alone or together with the software application.

The exercise training app processes the data and information from the sensors of the training gloves and provides data to the user, including predictive data regarding what that info is likely to read at when they use the alternative glove (i.e. weighted vs. non-weighted training glove, and/or gloves of various weights). The exercise training system is used for performance training especially for sports like basketball, football, martial arts, baseball, boxing, lacrosse, tennis, volleyball, and soccer. The sensors may be disposed on the fingertips, palms, backside of the hand and/or wrist without affecting or interfering with the exercise training.

The application may be in communication with one or more additional training devices via sensors and/or via control of such devices. Such devices may include but are not limited to training cones, hoops, goals, harriers, automated moving devices, opponent simulation devices, tracks, and the like and combinations thereof. Non-limiting examples of such devices include attaching sensors and/or control devices to one or more of the Dribblemac which may be found at http://globallsports.net/home.html and the Pop-Up Defender which may be found at http://popupdefender.com. Accordingly, the software application may combine/compile/analyze information received from such devices in concert with the information obtained from the wearable equipment (i.e. weighted and/or non-weighted gloves/equipment) to provide enhanced information and training.

According to one embodiment of the invention, there is shown an exercise training system that shows progress and improvement in exercise training to provide better training. The exercise training data shows progress in techniques, the data provides predictive information while training. The exercise training system provides additional data to trainers to allow them to improve training protocols and allows for better choices with regard to specific drills to perform and their durations of the exercise training.

According to one embodiment of the invention, there are two sets of wearable equipment, one is weighted and the other is non-weighted. Each set includes one or more onboard sensors that are in communication with a software application that performs predictive modeling based on data from use of the two sets of wearable equipment. The sensors may be in wireless communication with the software application and/or may include removable memory cards and/or access jacks through which data from the sensors may be provided to the software application. The software application may perform predictive modeling based on data projections, extrapolation techniques, interpolation techniques, and the like and combinations thereof. As a non-limiting example, the software application may establish a performance ratio for a particular characteristic (e.g. maximum speed of a bat swing) between the weighted a non-weighted wearable equipment (e.g. gloves) by observing that characteristic for a particular user over a period of time while using each of the weighted and non-weighted versions of the wearable equipment. The user may then practice with the weighted version for a period and when viewing statistics regarding practices, the software application may apply the performance ratio to also provide the user with a projected characteristic for that same activity while using non-weighted wearable equipment (e.g. the user continues to practice hat swings with the weighted gloves and gets a report/readout on the application that shows the actual maximum speed of the bat swing during their continued practicing and a projected maximum speed of the bat swing if using non-weighted gloves thus being able to see progress based on how they will perform in competition).

According to one embodiment of the invention, there is an exercise training system hat includes gloves, weights, sensors, and a software application. The software application includes a data processor for measuring speed, strength, impact, number of impacts, etc. The software application includes a predictive module and a feedback module. The feedback module shows the user how good they did vs. goals/expectations, i.e. are you dribbling with the right parts of your hand, is your left hand or right hand the more dominant hand, etc.).

According to one embodiment of the invention, there is shown an exercise training system that includes weights and sensors in/on training gloves. The exercise training system includes software wirelessly connected to the sensors of the training gloves. The exercise training system includes two sets of gloves, one weighted and one non-weighted training glove, with sensors and an exercise training application that does predictive modeling based on data from the two training gloves.

The software and/or wearable equipment may include information and/or device(s) sufficient to allow for the software to be able to tell from which equipment data is coming. As a non-limiting example, a sensor may include an identification code/number and may provide that to the software application along with data from the sensor. The software application may have registered that sensor according to its identification code/number as being associated with a particular set of wearable equipment, including but not limited to the weighted status of that equipment and/or other characteristics (e.g. type of equipment (glove/shoe/helmet), type of sensor, placement of sensor within the equipment). As another non-limiting example, the software may include a setup process to register new equipment with the software. As still another non-limiting example a sensor may include one or more selectable features that may be selectable via hardware settings (e.g. dip switches) and/or software settings within the sensor that may allow the user to use a sensor for multiple purposes (e.g. the user has one sensor that is selectably removable from each weighted glove and may be switched to various modes that allow for the software to know which glove it is in when it provides data).

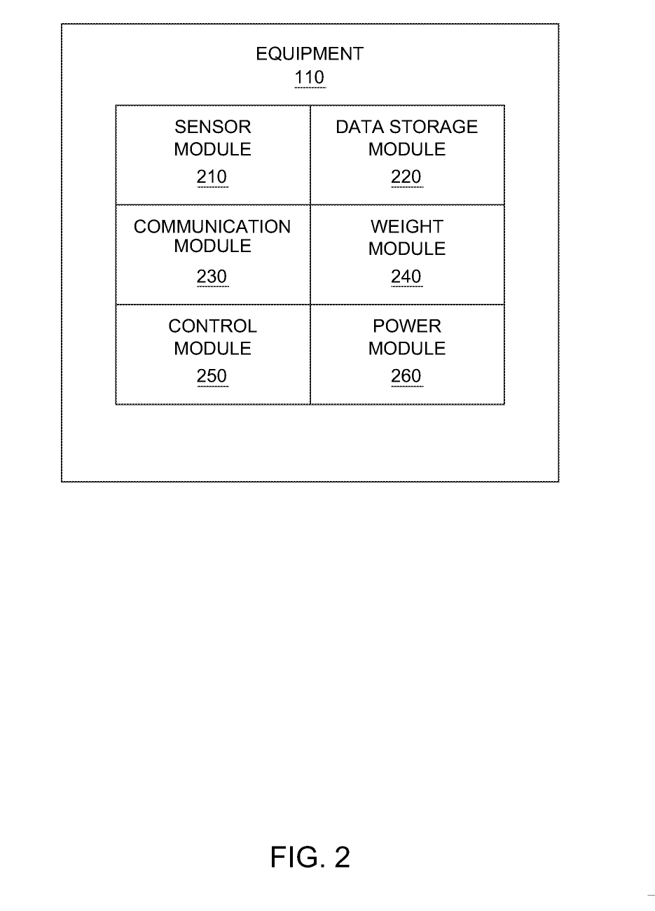

FIG. 2 is a module diagram of an equipment, according to one embodiment of the invention. There is shown equipment (e.g. a set of equipment) 110 including a sensor module 210, a data storage module 220, a. communication module 230, a weight module 240, a control module 250, and a power module 260. The illustrated equipment facilitates training of a user.

The equipment may be selected from the group of equipment consisting of: apparel, training aids, training tools, weights, exercise devices/systems, training facilities and the like and combinations thereof. The equipment may include one or more gloves, shoes, balls, weights, protective devices, movable barriers, goals, and the like and combinations thereof. It may be that the equipment includes two sets of equipment of a particular type, wherein the type may be, according to one non-limiting embodiment of the invention, selected from the group of types consisting of: gloves, shoes, belts, shoulder-pads, knee-pads, elbow-pads, helmets, wristbands, and shin-guards. The two sets of equipment of the particular type may include one set that is weighted (i.e. has a weight enhancement to increase the weight/resistance of the equipment) and one set that is not weighted (i.e. does not have a weight enhancement as compared to the weighted set).

The illustrated sensor module 210 includes one or more sensors for detecting one or more physical characteristics associated with the equipment and/or of a portion of the equipment (e.g. pressure on the palm as opposed to pressure on a fingertip), such as but not limited to pressure, velocity, acceleration, position, and the like and combinations thereof. It may be that the sensors are selected from the group of sensors consisting of: accelerometers, gyroscopes, photoelectric sensors, position sensors, tilt sensors, pressure sensors, temperature sensors, blood pressure sensors, heart rate monitors, and SpO2 sensors. It may be that each of the weighted equipment (e.g. a glove) and non-weighted equipment (e.g. a glove) includes a plurality of sensor types and such sensor types and placement of the same may be identical between the weighted and non-weighted equipment. Accordingly, similar performance data may be gathered from each of the weighted and non-weighted equipment. A sensor module may be as described in U.S. Pat. No. 6,593,732, issued to Dammkhler et al.; or a weight sensor module as described in U.S. Pat. No. 6,099,032, issued to Cuddihy et al. which is incorporated for their supported teachings herein. Non-limiting examples of a sensor module may be a sensor module as described in U.S. Pat. No. 6,593,732, issued to Dammkhler et al.; or a weight sensor module as described in U.S. Pat. No. 6,099,032, issued to Cuddihy et al. which is incorporated for their supported teachings herein.

Wherein there is a plurality of sensor types on the equipment, the system may be able to gather and analyze data more effectively/efficiently and/or may be able to more accurately predict performance for non-weighted equipment use. As a non-limiting example, wherein weighted and non-weighted gloves are utilized in ball handling training (e.g. football, basketball) and such gloves include accelerometers and pressure sensors. Performance data taken using the weighted and non-weighted gloves may allow the system to better correlate data points for acceleration and pressure to success/failure of ball handling between weighted and non-weighted glove use by providing at least two reference data sets (e.g. acceleration and pressure during ball handling) and may also be able to index/tag data sections (e.g. acceleration right before pressure indicates contact with the ball) as being particularly important/relevant during analysis.

The following are non-limiting examples of sensor hardware that may be part of one or more pieces of equipment utilized with one or more embodiments of the invention: micromachined capacitive accelerometers, piezoelectric resistive accelerometers, capacitive spring mass system base accelerometers, DC response accelerometers, servo force balance accelerometers, laser accelerometers, three collector pressure sensors e.g. piezoresistive strain gauges, capacitive, electromagnetic, piezoelectric, optical, and potentiometric), resonant pressure sensors, thermal pressure sensors, ionization pressure sensors, thermistors, thermocouples, resistance thermometers, silicon bandgap temperature sensors, inclinometer, tiltmeters, infrared sensors (e.g. as used in heart rate monitors), EKG monitors, light detectors (e.g. pulse oximeters), and the like and combinations thereof.

The illustrated data storage module 220 collects and stores sensor data and data associated therewith (e.g. time stamps, session ID, series ID). The data storage module is in communication with the modules and components of the system such that it and they may perform their intended functions. A data storage module may include a data storage device and may include one or more databases and/or data files. A memory storage device may be, but is not limited to, hard drives, flash memory, optical discs, RAM, ROM, and/or tapes. A non-limiting example of a data base is Filemaker Pro 11, manufactured by Filemaker 5261 Patrick Henry Dr., Santa Clara, Calif., 95054. Non-limiting examples of a data storage module may include: a HP Storage Works P2000 G3 Modular Smart Array System, manufactured by Hewlett-Packard Company, 3000 Hanover Street, Palo Alto, Calif., 94304, USA; or a Sony Pocket Bit USB Flash Drive, manufactured by Sony Corporation of America, 550 Madison Avenue, New York, N.Y., 10022.

The illustrated communication module 230 is functionally coupled to the other modules described herein such that they may each operate in their intended manners. The communication module may provide communication capabilities, such as wireless communication, to the modules and components of the system and the components and other modules described herein. The communication module may include physical component(s) such as but not limited to removable memory devices, cords, transponders, transceivers, and the like and combinations thereof. The communication module may provide communication between a wireless device, such as a mobile phone, and a computerized network and/or to facilitate communication between a mobile device and other modules described herein. The communication module may have a component thereof that is resident on a user's mobile device. Non-limiting examples of a wireless communication module may be but not limited to: a communication module described in U.S. Pat. No. 5,307,463, issued to Hyatt et al.; or a communication module described in U.S. Pat. No. 6,133,886, issued to Fariello et al., which are incorporated for their supported herein. It may be that each of the weighted and non-weighted equipment (e.g. gloves) includes a wireless communication module that transmits training data to a mobile computing device.

The illustrated weight module 240 is functionally coupled to one or more of the other modules/components herein such that each are able to perform their intended functions. The weight module may include information regarding a weighted vs. unweighted status of a particular piece of equipment. The weight module may include physical components that enhance a weight of an equipment, such as but not limited to one or more weight bodies disposed in closed pockets within the weighted equipment (e.g. wearable e.g. gloves). The weight module may include one or more sensors that may detect the presence and/or type of weight bodies that may be coupled to and/or disposed inside the equipment. The weight module may simple include an indicator signal/data that indicates the status of the equipment as being either weighted or unweighted and if weighted it may include information regarding the amount of weight disposed therewith.

The illustrated control module 250 provides operational instructions and commands to the modules and components of the system. The control module is in communication with the modules and components of the system (and/or other modules described herein) and provides managerial instructions and commands thereto. The source of such instructions/commands may be from one or more other modules described herein and/or through interactions between one or more other modules described herein. The control module sets parameters and settings for each module and component of the system. Non-limiting examples of a control module may be a control module described in U.S. Pat. No. 5,430,836, issued to Wolf et al.; or a control module described in U.S. Pat. No. 6,243,635, issued to Swan et al. which are incorporated for their supporting teachings herein. A control module may include but is not limited to a processor, a state machine, a script, a decision tree, and the like.

The illustrated power module 260 provides power to the equipment as needed. It is in functional communication with the other components/modules described herein to the degree that each is able to perform its expected functions. The power module may include one or more power supplies and/or batteries to provide electrical power. There may be one or more power control devices/circuits that regulate power distribution/delivery. There may be power conduits functionally coupled to the components such that power may be distributed thereto. Non-limiting examples of power modules may be described in U.S. Pat. Nos. 6,362,980; 4,652,769; and 6,987,670; and U.S. Patent Application No. 2009/0,153,477, which are incorporated herein for their supporting teachings. The power module may include one or more power generation devices, such as but not limited to solar cells (photovoltaic and/or thermoelectric devices), electromagnetic induction circuits, static electricity gathering circuits, electrochemical extraction devices, piezo electric power gathering circuits, and the like and combinations thereof.

Advantageously, the illustrated equipment 110 may be utilized by a user in training activities and physical parameter data may be detected and recorded in association therewith, both in training with weighted equipment and non-weighted. equipment. Accordingly, performance may be observed by the system in both weighted and non-weighted situations and preserved for future analysis.

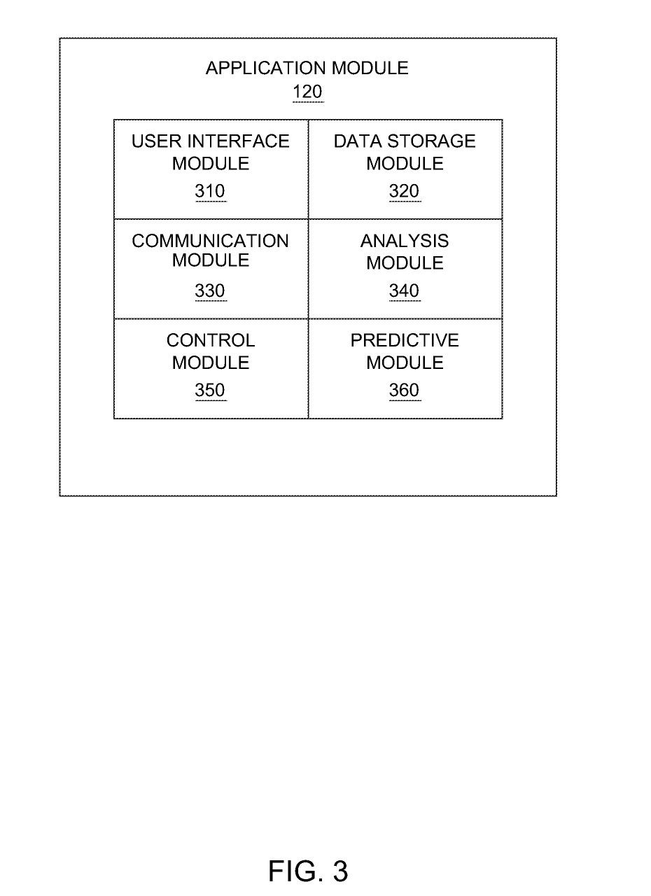

FIG. 3 is a module diagram of an application module, according to one embodiment of the invention. There is shown an application module 120 that includes a user interface module 310, a data storage module 320, a communication module 330, an analysis module 340, a control module 350, and a predictive module 360. The illustrated application module 120 provides a useful user interface for interfacing with data collected through operation of the equipment 110.

The illustrated user interface module 310 provides a user interface for interaction with the application module by a user wherein the user is able to view data and information associated therewith. The user interface module includes a display and/or other sensory projection device (e.g. speaker) such that the user may be able to experience/detect information provided therethrough. The display may be an LCD display, such as that of a smartphone/laptop/tablet device. The user interface module includes instructions for displaying information and for receiving user input such as but not limited through a touchscreen that may be integrated with the display. The user interface may allow the user make selections, to change how data is displayed, to change what data is displayed, to adjust settings, and the like and combinations thereof. The user interface may include one or more GUI (graphical user interface), one or more display devices, one or more libraries of communication protocols, one or more libraries of communication image styles (e.g. font libraries, skins), and one or more user input interpretation protocols that allows the user interface to receive and understand commands by a user. Non-limiting examples of user interface modules include operating systems (e.g. MAC iOS, Windows, Android) and those taught by U.S. Pat. Nos. 7,185,290; 5,903,881; 6,956,593; and 7,027,101, which are incorporated herein for their supporting teachings.

Such may include one or more user interface modules or devices that may be embodied in software instructions for controlling display on a display (such as but not limited to a TV, monitor, computer, cell phone/tablet screen, holographic display, etc.) and/or for routing signals from an input device (such as but not limited to a keyboard, touchscreen, mouse, etc.) such that a user may perform exercise data entries or queries in the computerized system, search suggestions or recommendations, and receive exercise data information therefrom. Such may be embodied in one or more user interfaces that permit browsing of the computerized system. Such may be embodied in one or more user interfaces that permit users to make adjustments, changes, and otherwise provide personal profile or account updates to the computerized system. Such may be embodied in one or more user interfaces that permit review of data from the system, such as but not limited to exercise training data, interactive data, user and usage data, management data, database usage, record data, etc. Non-limiting examples of a user interface module may be a HTML player, client server application, Java script application. A non-limiting example of a user interface module may be a FlowPlayer 3.1, manufactured by FlowPlayer LTD, Hannuntic 8 D, ESPOO 02360, Helsinki, Finland. Non-limiting examples of a user interface module may be a display/interface module as described in U.S. Pat. No. 6,272,562, issued to Scott et al.; a touch screen interface module as described in U.S. Pat. No. 5,884,202 and U.S. Pat. No. 6,094,609, issued to Arjomand, which are incorporated for their supporting teachings herein.

The illustrated data storage module 320 collects and stores sensor data and data associated therewith (e.g. tune stamps, session ID, series ID). The data storage module is in communication with the modules and components of the system such that it and they may perform their intended functions. The data storage module is configured to store exercise training data, along with personal user goals, data and profiles. In addition, the data storage module is configured to store various metadata generation and tagging commands to the system for use. A data storage module may include a data storage device and may include one or more databases and/or data files. A memory storage device may be, but is not limited to, hard drives, flash memory, optical discs, RAM, ROM, and/or tapes. A non-limiting example of a data base is Filemaker Pro 11, manufactured by Filemaker Inc., 5261 Patrick Henry Dr., Santa Clara, Calif., 95054. Non-limiting examples of a data storage module may include: a HP Storage Works P2000 G3 Modular Smart Array System, manufactured by Hewlett-Packard Company, 3000 Hanover Street, Palo Alto, Calif, 94304, USA; or a Sony Pocket Bit USB Flash Drive, manufactured by Sony Corporation of America, 550 Madison Avenue, New York, N.Y., 10022.

The illustrated communication module 330 is functionally coupled to the other modules described herein such that they may each operate in their intended manners. The communication module may provide communication capabilities, such as wireless communication, the modules and components of the system and the components and other modules described herein. The communication module may include physical component(s) such as but not limited to removable memory devices, cords, transponders, transceivers, and the like and combinations thereof. The communication module may provide communication between a wireless device, such as a mobile phone, and a computerized network and/or to facilitate communication between a mobile device and other modules described herein. The communication module may have a component thereof that is resident on a users mobile device. Non-limiting examples of a wireless communication module may be but not limited to: a communication module described in U.S. Pat. No. 5,307,463, issued to Hyatt et al.; or a communication module described in U.S. Pat. No. 6,133,886, issued to Fariello et al., which are incorporated for their supported herein. It may be that each of the weighted and non-weighted equipment (e.g. gloves) includes a wireless communication module that transmits training data to a mobile computing device.

The illustrated analysis module 340 receives and processes data received from the equipment. The illustrated analysis module is functionally coupled to other modules and components of the system as appropriate for each to perform their various functions. The analysis module may store received data within one or more data structures, may assign metadata to the same (e.g. session ID, account ID), may incorporate user input in association with the received data (e.g. success/fail indicators in association with training activities), may mathematically fit curves to data, may replace data sets with mathematical representations, may collate data, may compare data sets, may associate data sets with each other, may match new data sets to old data sets, and/or may perform one or more data cleaning, manipulation, transformation, translation, and/or aggregation protocols on received/stored data. Non-limiting examples of analysis modules are described in U.S. Pat. Nos. 7,729,789; 6,270,457; 6,567,536; and U.S. Application No. 2008/0,212,866, which are incorporated herein for their supporting teachings.

In another non-limiting embodiment, there is a training system having an analysis module that includes the data processor, a data storage module functionally coupled to the analysis module such that the analysis module may call data therefrom, and a user interface module functionally coupled to the data processor module such that analysis therefrom may be reported to the user interface module on demand from a user.

The illustrated control module 350 provides operational instructions and commands to the modules and components of the system. The control module is in communication with the modules and components of the system (and/or other modules described herein and provides managerial instructions and commands thereto. The source of such instructions/commands may be from one or more other modules described herein and/or through interactions between one or more other modules described herein. The control module sets parameters and settings for each module and component of the system. Non-limiting examples of a control module may be a control module described in U.S. Pat. No. 5,430,836, issued to Wolf et al.; or a control module described in U.S. Pat. No. 6,243,635, issued to Swan et al. which are incorporated for their supporting teachings herein. A control module may include but is not limited to a processor, a state machine, a script, a decision tree, and the like.

The illustrated predictive module 360 utilizes data, processed/analyzed or otherwise, and generates predictive information, such as but not limited to predictive models, predictions of performance data, and/or predictions of performance. The predictive module is functionally coupled to other components and modules described herein such that each may perform their expected functions. The predictive module may take performance data associated with weighted and non-weighted equipment usage and use that information to form predictive models for a particular user and/or for particular equipment such that future performance data may be compared to the model to determine untested performance data, such as but not limited to predicting non-weighted performance with particular equipment based on performance observed with weighted equipment. Such may also be utilized to predict recovery/improvement progress over time while using particular sets of weighted/non-weighted equipment based on performance progress made by others and/or performance progress made by a particular user and/or using particular equipment. Such may be accomplished by fitting curves to sets of performance data, weighting data, fitting/generating a function to map performance on weighted equipment to expected performance on non-weighted equipment using one or more of the following techniques: polynomial regression, polynomial interpolation, function fitting using least-squares techniques, Deming regression, orthogonal regression, and the like and combinations thereof. Non-limiting examples of predictive modules (e.g. predictive modeling) are taught in U.S. Pat. No. 7,283,982; and U.S. Patent Application Nos. 2010/0,081,971; and 2009/0,183,218, which are incorporated by reference herein for their supporting teachings.

In one non-limiting embodiment, instructions of a data processor that may be part of an application module may include instructions for generating predictive information about how a user will currently perform using the non-weighted wearable equipment based on generating a mapping rule by comparing historical data for that user from both the weighted wearable equipment sensor and the non-weighted wearable equipment sensor and by applying the mapping rule to current sensor data from the weighted wearable equipment sensor. Such may be accomplished by receiving and recording historical data of performance of a user (e.g. motion information of the equipment, pressure information, health information of the user) through sensors disposed on both weighted and non-weighted equipment of the same type and associating such performance data where it is co-extensive in time used the weighted and non-weighted equipment on the same day and therefore performance should be analogous between each). A pattern may be determined automatically by the system that associates how well the user does with weighted equipment as compared to non-weighted equipment. Such a pattern may be expressed as a simple function that maps weighted performance to non-weighted performance. As a non-limiting example, acceleration data may be recorded while using a glove, weighted and non-weighted, and it may be observed that peak acceleration of the non-weighted glove tends to be about 125% of that of the weighted glove. Accordingly, the system may automatically generate a function f(x)=1.25*x wherein x is the peak acceleration observed with the weighted glove and f(x) is the expected peak acceleration while using a non-weighted glove. The system may therefore, on receiving performance data with the weighted glove, output expected performance data with the non-weighted glove. This expected performance data may be matched against a threshold of performance data that may be stored within the system and such a matching may be displayed through a user interface. Where the threshold performance data is a goal for peak acceleration, or other performance, the user may be able to continue practicing with the weighted equipment and not continually testing performance using the non-weighted equipment, yet still see predicted performance against the non-weighted goal.

It may be that there are instructions for generating predictive performance data that include instructions for generating a mapping rule by comparing historical data for that user from both the weighted glove and the non-weighted glove and by applying the mapping rule to current accelerometer data from the weighted glove. Such data may be marked with specific markers associated with particularly relevant events (e.g. contact with a ball, peak acceleration, activation of pressure sensors, peak hear ate) and that such particularly marked performance data may be mapped against similarly marked historical data and the mapped according to the mapping rule to determine mapped data, which may correspond to predicted performance using different equipment. A mapping rule may be as simple as a ratio to apply to data to change it from actual performance data to mapped performance data, or a more complicated function-based, table-based, and/or rule-based mapping may be performed on the data.

One or more of the following techniques associated with predictive modeling may be incorporated within the predictive module: regression models, parametric models, non-parametric models, semi-parametric models, group method of handling data, Naïve Bayes, k-nearest neighbor, majority classifier, support vector machines, random forests, boosted trees, classification and regression trees (CART), multivariate adaptive regression splines (MARS), neural networks, ACE, AVAS, ordinary least square, generalized linear models (GLM), logistic regression, generalized assistive models, robust regression, and/or semiparametric regression. The predictive module may include one or more formulas, scripts, algorithms, data pools, and the like and combinations thereof that may facilitate in generating predictive information based on observed characteristics/levels from sensors. Reporting may be via print-outs, on-screen presentation of data, color-coded lights or other displays on the wearable equipment (e.g. LED on the wearable equipment is red and changes to green once a threshold of predictive performance is reached), and the like and combinations thereof. Predictive information may be displayed as simple data, nomograms, point estimates, tree-based methods, score charts, charts, graphs, pictographs, and the like and combinations thereof. Non-limiting examples of a predictive module may be a data analysis system as described in U.S. Patent Publication No.: 2012/0290576; or an analysis system as described in U.S. Patent Publication No.: 2011/0208519, which are incorporated for their supporting teachings herein.

It may be that there is a user interface module disposed on a portable computing device that may be in functional communication with a data processor such that a user of the portable computing device can receive predictive performance data therefrom once/as the predictive performance data is generated by the predictive module. Such a user interface may include one or more athlete accounts that may be different from a user interface for a coach account and wherein each of the athlete account and the coach account can access the same set of training and predictive data over different mobile computing devices.

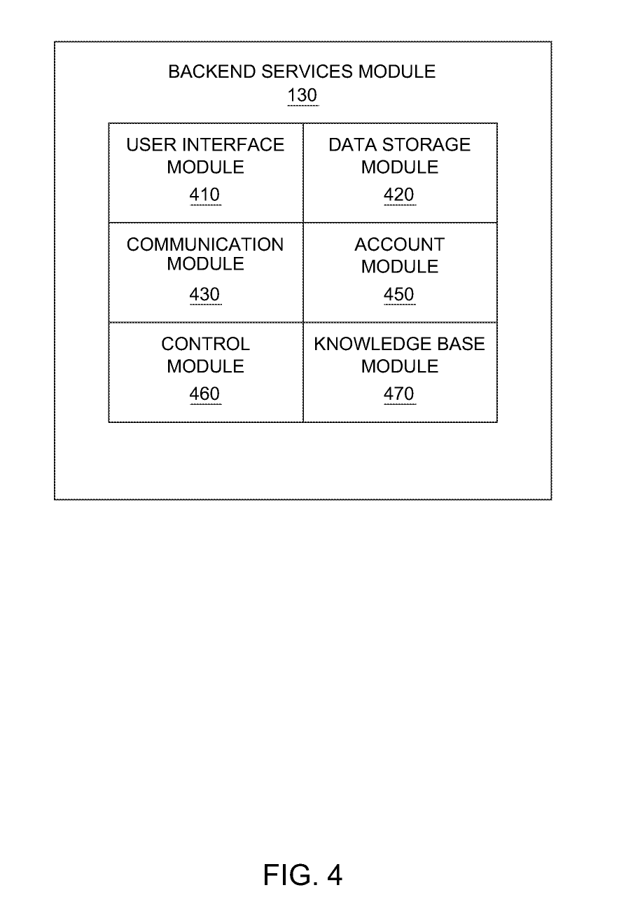

FIG. 4 is a module diagram of a backend services module, according to one embodiment of the invention. There is shown a back-end services module 130 that includes a user interface module 410, a data storage module 420, a communication module 430, an account module 450, a control module 460, and a knowledge-base module 470. The illustrated back-end service module 130 provides management of the training system and allows for the same training system to service a multiplicity of users. The illustrated user interface module 410 provides a user interface for interaction with the application module by a user wherein the user is able to view data and information associated therewith. The user interface module includes a display and/or other sensory projection device (e.g. speaker) such that the user may be able to experience/detect information provided therethrough. The display may be an LCD display, such as that of a smartphone/laptop/tablet device. The user interface module includes instructions for displaying information and for receiving user input such as but not limited through a touchscreen that may be integrated with the display. The user interface may allow the user make selections, to change how data is displayed, to change what data is displayed, to adjust settings, and the like and combinations thereof. The user interface may include one or more GUI (graphical user interface), one or more display devices, one or more libraries of communication protocols, one or more libraries of communication image styles (e.g. font libraries, skins), and one or more user input interpretation protocols that allows the user interface to receive and understand commands by a user. Non-limiting examples of user interface modules include operating systems (e.g. MAC iOS, Windows, Android) and those taught by U.S. Pat. Nos. 7,185,290; 5,903,881; 6,956,593; and 7,027,101, which are incorporated herein for their supporting teachings.

Such may include one or more user interface modules or devices that may be embodied in software instructions for controlling display on a display (such as but not limited to a TV, monitor, computer, cell phone/tablet screen, holographic display, etc.) and/or for routing signals from an input device (such as but not limited to a keyboard, touchscreen, mouse, etc.) such that a user may perform exercise data entries or queries in the computerized system, search suggestions or recommendations, and receive exercise data information therefrom. Such may be embodied in one or more user interfaces that permit browsing of the computerized system. Such may be embodied in one or more user interfaces that permit users to make adjustments, changes, and otherwise provide personal profile or account updates to the computerized system. Such may be embodied in one or more user interfaces that permit review of data from the system, such as but not limited to exercise training data, interactive data, user and usage data, management data, database usage, record data, etc. Non-limiting examples of a user interface module may be a HTML player, client server application, Java script application. A non-limiting example of a user interface module may be a FlowPlayer 3.1, manufactured by FlowPlayer LTD, Hannuntie 8 D, ESPOO 02360, Helsinki, Finland. Non-limiting examples of a user interface module may be a display/interface module as described in U.S. Pat. No. 6,272,562, issued to Scott et al.; a touch screen interface module as described in U.S. Pat. No. 5,884,202 and U.S. Pat. No. 6,094,609, issued to Arjomand, which are incorporated for their supporting teachings herein.

The illustrated data storage module 420 collects and stores sensor data and data associated therewith (e.g. time stamps, session ID, series ID). The data storage module is in communication with the modules and components of the system such that it and they may perform their intended functions. A data storage module may include a data storage device and may include one or more databases and/or data tiles. A memory storage device may be, but is not limited to, hard drives, flash memory, optical discs, RAM, ROM, and/or tapes. A non-limiting example of a data base is Filemaker Pro 11, manufactured by Filemaker Inc., 5261 Patrick Henry Dr., Santa Clara, Calif., 95054. Non-limiting examples of a data storage module may include: a HP Storage Works P2000 G3 Modular Smart Array System, manufactured by Hewlett-Packard Company, 3000 Hanover Street, Palo Alto, Calif., 94304, USA; or a Sony Pocket Bit USB Flash Drive, manufactured by Sony Corporation of America, 550 Madison Avenue, New York, N.Y., 10022.

The illustrated communication module 430 is functionally coupled to the other modules described herein such that they may each operate in their intended manners. The communication module may provide communication capabilities, such as wireless communication, to the modules and components of the system and the components and other modules described herein. The communication module may include physical component(s) such as but not limited to removable memory devices, cords, transponders, transceivers, and the like and combinations thereof. The communication module may provide communication between a wireless device, such as a mobile phone, and a computerized network and/or to facilitate communication between a mobile device and other modules described herein. The communication module may have a component thereof that is resident on a user's mobile device. Non-limiting examples of a wireless communication module may be but not limited to: a communication module described in U.S. Pat. No. 5,307,463, issued to Hyatt et al.; or a communication module described in U.S. Pat. No. 6,133,886, issued to Fariello et al., which are incorporated for their supported herein. It may be that each of the weighted and non-weighted equipment (e.g. gloves) includes a wireless communication module that transmits training data to a mobile computing device.

The illustrated account module 450 manages accounts for a multiplicity of users. The account module is functionally coupled to other components and modules described herein such that each may serve their intended functions. The account module may perform one or more of the following functions: new account creation, account settings management, account data association/storage, managing account permissions, associated related accounts, account deletion, account activation/deactivation, account sharing and combinations thereof. The following teach non-limiting examples of account modules and account module functions and are incorporated by reference for their supporting teachings: U.S. Patent Application Nos. 2012/0,078,735; 2011/0,302,083; and 2008/0,195,741; and U.S. Pat. No. 7,433,710.

The illustrated control module 460 provides operational instructions and commands to the modules and components of the system. The control module is in communication with the modules and components of the system (and/or other modules described herein) and provides managerial instructions and commands thereto. The source of such instructions/commands may be from one or more other modules described herein and/or through interactions between one or more other modules described herein. The control module sets parameters and settings for each module and component of the system. Non-limiting examples of a control module may be a control module described in U.S. Pat. No. 5,430,836, issued to Wolf et al.; or a control module described in U.S. Pat. No. 6,243,635, issued to Swan et al. which are incorporated for their supporting teachings herein. A control module may include but is not limited to a processor, a state machine, a script, a decision tree, and the like.

The illustrated knowledge base module 470 stores performance data received by the system and facilitates in increasing the accuracy and capabilities of the predictive module. The knowledge base module 470 is functionally coupled to other modules and components described herein such that each may perform their intended functions. The knowledge base module may store performance data in association with data regarding which equipment was used, by which user, at which time, under which circumstances (e.g. if the athlete was injured, particularly encumbered, undergoing a specific type of training), used in which activities/training/exercise, and the like and combinations thereof such that similarly received data may be matched against a large library of stored performance data. Such may also allow for particular application modules to receive initial mapping functions with respect to particular equipment/sets so that predictions may be made earlier than usual, wherein little or no data for a particular user with a particular set of equipment may be yet gathered. The knowledge base may also be automatically consulted by the application module to trouble-shoot or determine the source of outlier information. As a non-limiting example, performance data of weighted and non-weighted equipment may be matched against stored information within the knowledge base upon realizing that the actual performance data received in association with a particular user does not map similarly to how the knowledge base would expect the function mapping to occur. Such contrary mapping may be compared to a large library of historical mapping for a wide range of users with the same equipment. Where a similar match is made by the system, the users may be able to identify particular circumstances, conditions, difficulties, injuries and the like that may be revealed by finding data sets that have more in common with the observed data than with the typical/average data within the system. The following teach knowledge base systems/modules and are incorporated by reference for their supporting teachings: U.S. Pat. Nos. 5,107,499; and 6,220,743; and U.S. Patent Application Nos. 2005/0,044,110; 2002/0,188,622; and 2004/0,122,707.

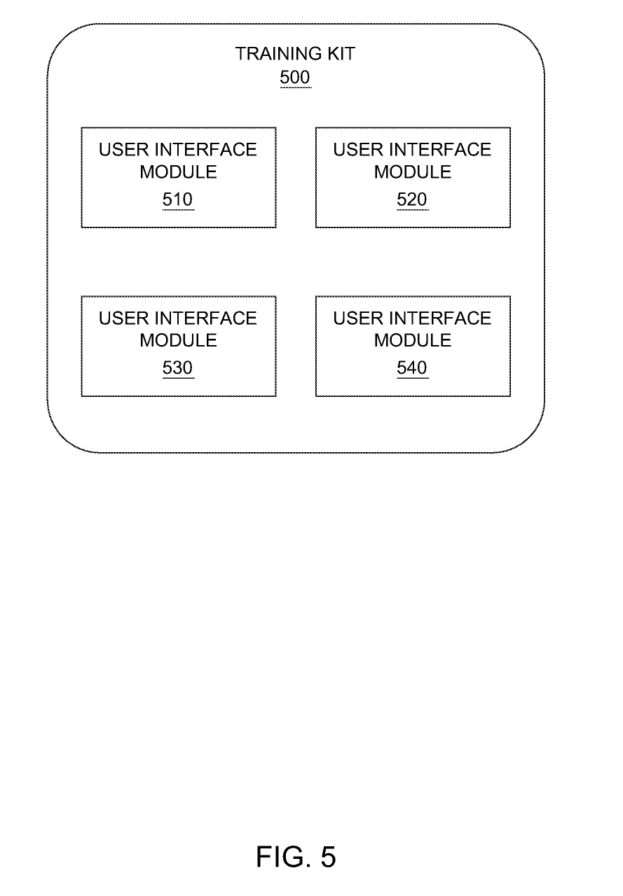

FIG. 5 is a module diagram of a training kit, according to one embodiment of the invention. There is shown a training kit 500 including equipment 510, instructions 520, software 530, and training accessories 540. The illustrated kit is configured to facilitate training by enabling a user to utilize various equipment in an efficient and effective manner by taking advantage of predictive modeling that reduces the amount of data needed to be acquired and/or coaching required to gain performance advantages from various sets of equipment, and especially weight-enhanced equipment, such as but not limited to weighted gloves. The kit may be provided all together in a single container or access to portions of the kit may be provided in various modes (e.g. the kit may include a link with a password to download the software and/or instructions and/or instructions to generate a user account where such may be acquired online).

The illustrated equipment 510 may include training equipment such as but not limited to training apparel, training devices, exercise devices, sports equipment and the like such as but not limited to gloves, shoes, body protection devices, apparel, balls, bats, nets, pucks, sticks, harnesses, and the like and combinations thereof. The equipment within the kit may include two or more of each type of equipment with one being weight enhanced and the other not. Alternatively, the equipment may include structures for allowing the equipment to be selectably and reversibly modified to be weight enhanced, such that the user can use the weight enhanced version for a time and then modify the equipment to be non-weight enhanced and vice-versa. The equipment includes one or more sensors incorporated therein and/or disposed thereon for recording motion, pressure, temperature, health, or other information relevant to performance of the user with respect to the equipment or with respect to one or more activities involving the equipment.

The illustrated instructions 520 provide information for the user in how to utilize the kit. Such may include care and use instructions for the equipment, instructions on how to modify the equipment (e.g. change from weighted to non-weighted), how to install and use the software associated therewith, information about the sensors, instructions for activities to perform with the equipment, instructions on how to record and/or annotate performance data, how to initialize one or more of the modules described herein, and/or how to otherwise benefit from the kit. The instructions may be presented in written form (e.g. booklet) and/or may be presented electronically (e.g. How To instructions that come with a downloadable application). The instructions may include various sections associated with various types of users (e.g. coach instructions, athlete instructions, sys admin instructions).

The illustrated software 530 provides the capability to collect, store and process sensor information from the equipment during use. The software may be a downloadable application to be installed on a smartphone, tablet, pc, laptop, smartwatch or the like and combinations thereof. Such may be provided by download over a network and/or by storage on a fixed medium (e.g. flashdrive, USB thumb drive, DVD/CD-ROM). The software may include one or more of the modules described herein, especially module associated with the application module.

The illustrated training accessories 540 may include one or more items that facilitate operation of the equipment and/or software. Such may include but is not limited to: grips, tape, measurement tools, weights, marking devices, wraps, balls, practice guides, coaching materials/media, targets, obstacles, timers, whistles, lotions, adhesives, and the like and combinations thereof.

There may be a training kit that includes one or more of the following: a weighted wearable equipment of a type having a sensor and including a weight enhancement; a non-weighted wearable equipment of the same type as the weighted wearable sensor, the non-weighted wearable equipment having a sensor and not including a weight enhancement; and/or instructions for accessing a training application that can analyze data received from the sensor of each of the weighted wearable equipment and the non-weighted wearable equipment and/or can generate predictive information derived from exercise training data from the sensors. It may be that the equipment type is a glove wherein the sensor of each glove is an accelerometer.

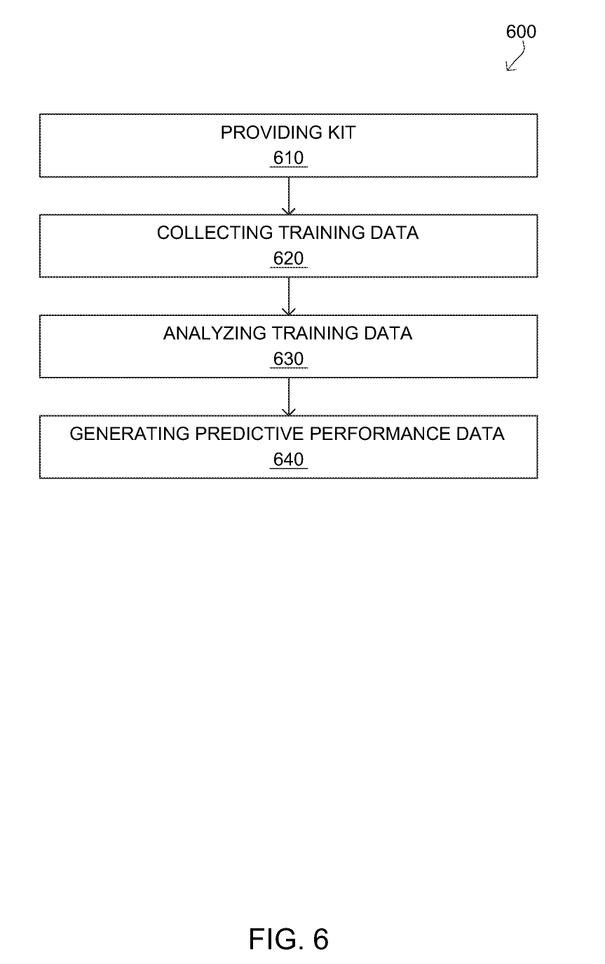

FIG. 6 is a flowchart of a method of training according to one embodiment of the invention. The illustrated method includes providing a training kit 610, collecting training data 620, analyzing training data 630, and generating predictive performance data 640. It is understood that other steps/processes/methods/activities described herein may augment/supplement the description of this method and that such are specifically contemplated within this application.

In operation, the method of training allows a user to benefit from enhanced training equipment that speeds up the training process and allows for more accurate prediction of the benefits of use of such equipment and the timing of how and when those benefits will be achieved. It also reduces the time required to gain such benefits.

The illustrated method includes the step of providing a kit, such as the kit described in FIG. 5, wherein weighted equipment and non-weighted equipment, each having sensors functionally coupled to an application module, are provided to a user for use in training activities.

The illustrated method includes the step of collecting may include collecting weighted training data for a user from use of equipment. The equipment may include weighted wearable equipment of a type having a sensor and including a weight enhancement. The user would utilize the equipment and performance data associated with such use would be collected automatically.

The illustrated step of collecting may also include collecting non-weighted training data for the user from a non-weighted wearable equipment of the same type as the weighted wearable sensor, the non-weighted wearable equipment having a sensor and not including a weight enhancement. The user would utilize the equipment and performance data associated with such use would be collected automatically.

The step of analyzing may include analyzing weighted and non-weighted training data for the user in combination with information about a weight differential between the weighted wearable equipment and the non-weighted wearable equipment. The information about the weight differential may include an actual weight differential or just that there is a weight differential. The data analysis may include fitting a function to the data points; relating the weighted and non-weighted data points to each other by a mapping function, table, or otherwise; and/or by associating collected data with data stored in a knowledge base or with predetermined mapping functions, tables, or otherwise. Accordingly, the collected data has context that has either been calculated or matched and therefore carries meaning beyond just its historical record-keeping significance.

The step of generating predictive performance data may include generating such for the user derived from analyzing the weighted and non-weighted training data. Such may be carried out by applying a mapping function, table, rule, or otherwise to a set of collected data. The predictive information may predict one or more aspects of uncollected information, such as but not limited to predicting: a course of expected progress by utilizing a training scheme, performance capabilities while using equipment that is different weighted or non-weighted) as compared to collected data, and the like and combinations thereof.

As a non-limiting example, a user suffering from a particular injury may utilize the equipment, thereby collecting data during such use. The collected data may be analyzed and thereby matched against similar data in a knowledge base to data about injury recovery of others having similar injuries with similar collected data, and thus a prediction may be made about the time of recovery.

As another non-limiting example, a user may utilize weighted and non-weighted equipment in training and may thereby accumulate a body of performance data specific to that user. The user may then reduce use of the non-weighted equipment to a minimal or zero amount, and continue accumulating data with regards to the weighted equipment while receiving reporting that predicts performance capabilities with non-weighted equipment which is then compared against a goal for desired performance, without having to waste training time on testing with non-weighted equipment.

Advantageously, the user of such a system may be able to automatically receive predictive performance data that is customized to that specific individual and relevant to their particular abilities at a given moment, while benefiting from enhanced training techniques using weighted wearable equipment.

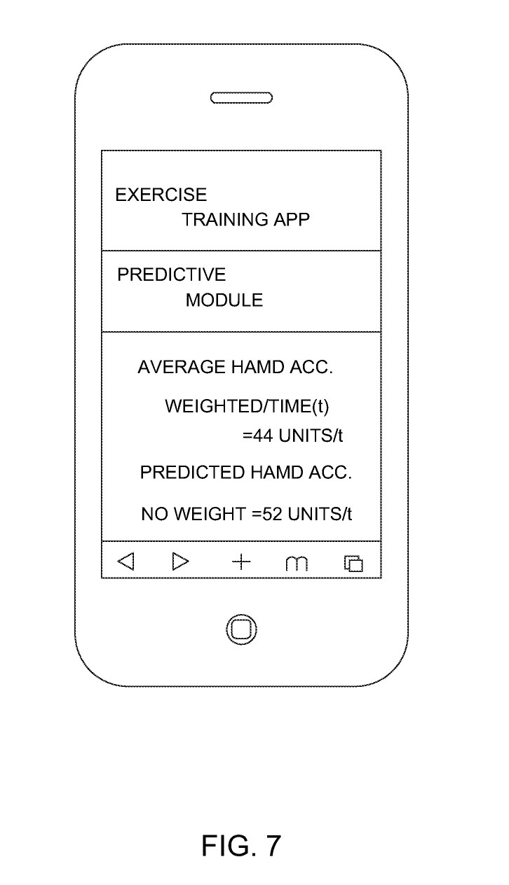

FIG. 7 is a prophetic view of a screen of a smartphone displaying predictive information based on sensor data, according to one embodiment of the invention. There is shown a screen of a smartphone including a display. On the display is information related to collected acceleration information using weighted equipment in association with predicted acceleration information using non-weighted equipment. The prophetic numbers are expressed in generic units per tune and do not relate to any specific measurements taken by Applicant.

Advantageously, the user may, in real-time, train with weighted equipment and then immediately receive predictive information in a convenient manner over their smartphone. This allows them the benefits of the weighted equipment without having to estimate or guess how well they would perform with non-weighted equipment, such as but not limited to in a competitive situation.

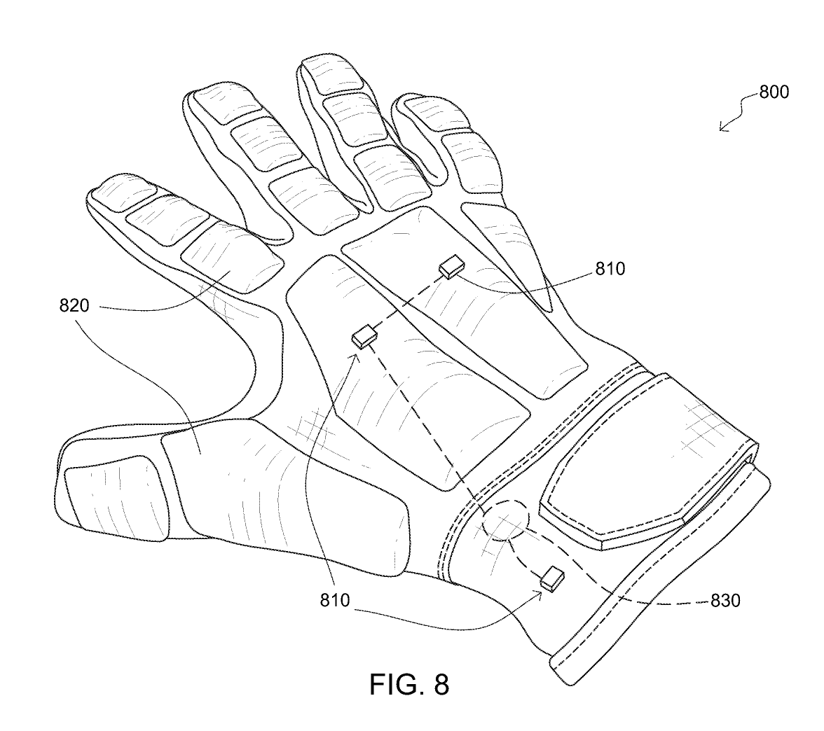

FIG. 8 is a top perspective view of a weighted training glove with sensors of an exercise training system, according to one embodiment of the invention. There is shown a weighted training glove 800 including a plurality of sensors 810 disposed about a backside of the glove and wrist along with weight filled closed pockets 820 that enhance the weight of the gloves 800.

According to one embodiment of the invention, there is shown a non-weighted training glove to provide a control example in respect to the weighted training glove. The non-weighted training glove includes a plurality of sensors disposed about a backside thereof. The illustrated sensors are disposed about the backside of the hand and the wrist and configured to gather exercise training data. The illustrated sensors are coupled to each other and to a wireless communication module 830 embedded in the wrist of the gloves so that sensor data therefrom may be communicated to one or more devices/systems outside the gloves, such as but not limited to an application module.

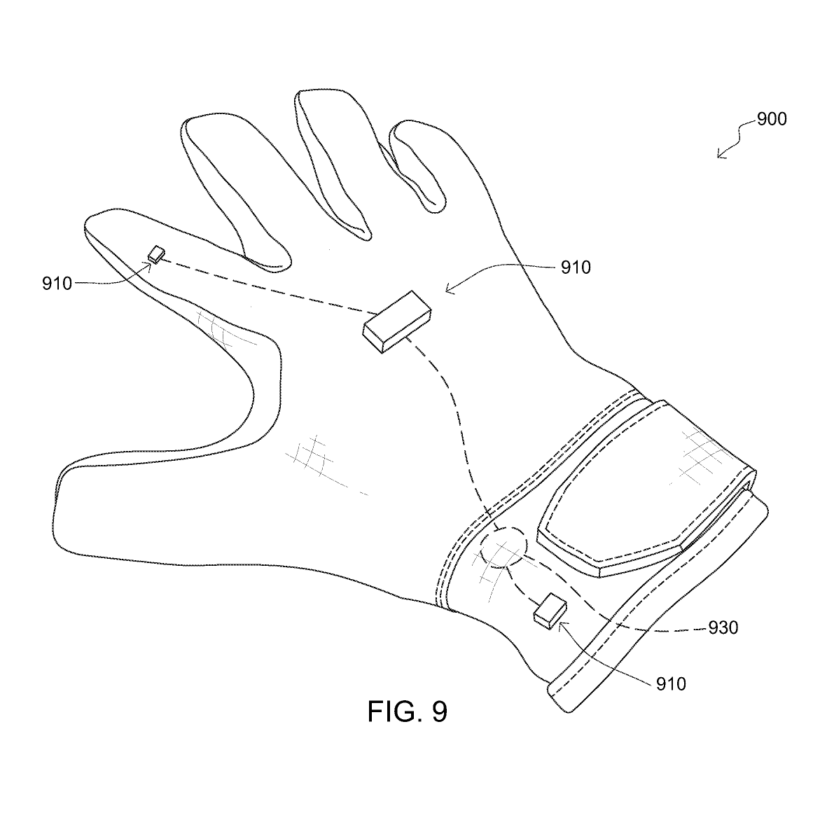

FIG. 9 is a top perspective view of a non-weighted glove with sensors of an exercise training system, according to one embodiment of the invention. There is shown a training glove 900 including a plurality of sensors 910 disposed about a finger region and back-hand portion of the training glove 900.

According to one embodiment of the invention, there is shown a non-weighted training glove non-weighted as compared to the weighted glove of FIG. 8). The illustrated training glove includes a plurality of sensor disposed about an exterior surface of the weighted training glove. The sensors are configured to gather exercise training data of the user while performing an exercise or sport activity. The illustrated sensors 910 are coupled to each other and to a wireless communication module 930 embedded in a wrist portion of the glove.

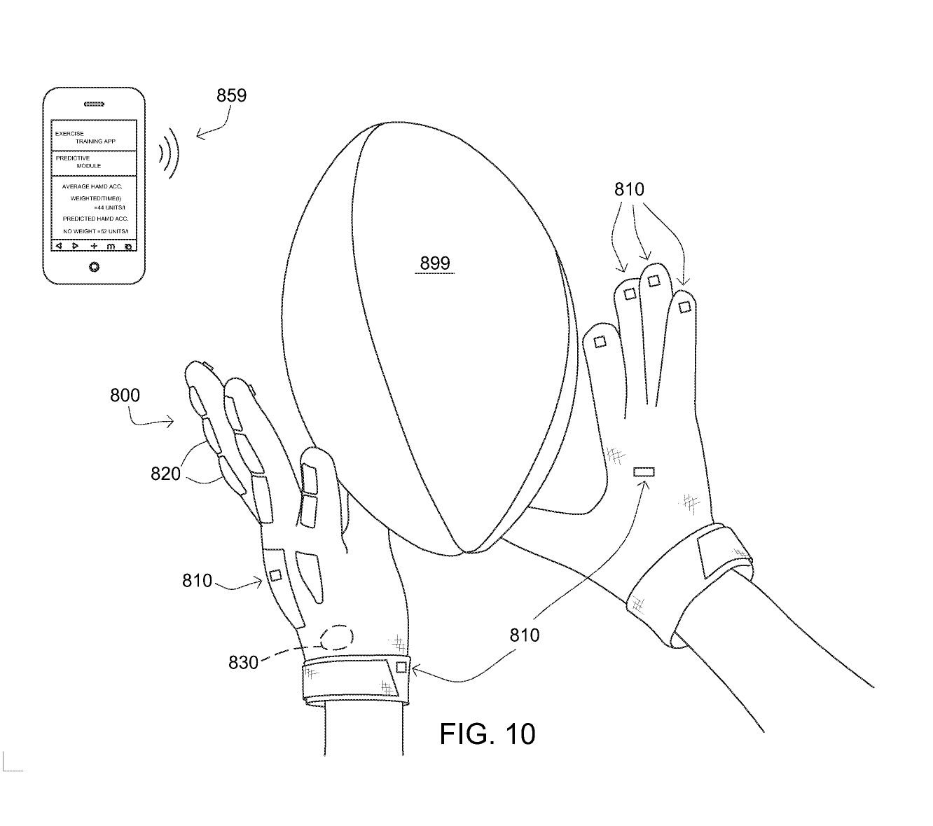

FIG. 10 is a perspective view of a pair of weighted gloves of an exercise training system about to catch a football, according to one embodiment of the invention. There is shown a pair of training gloves 800 including a plurality of sensors 810 about to catch a football 899.

According to one embodiment of the invention, there is shown an exercise training system including a pair of weighted training gloves 800 each having a plurality of sensors 810 disposed thereon. The sensors 810 are disposed about the fingertips, palm, backside of the hand, and wrist regions of the weighted training gloves 800. The sensors are in wireless communication with a mobile device 859 through a wireless communication module 830 that is functionally coupled to the sensors 810t, wherein the mobile device 859 includes an exercise training app stored therein and configured to gather and analyze exercise training data of the user while performing an exercise or sports activity. The exercise training app gathers exercise training data from the sensors to determine physical characteristics, traits, attributes from each glove to determine or predict exercise performance data about the user when the user is wearing a weighted glove, the non-weighted glove, or no training glove at all.

It is understood that the above-described embodiments are only illustrative of the application of the principles of the present invention. The present invention ay be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A training system over a computerized network, comprising:
a) a weighted wearable equipment of a type having a sensor and including a weight enhancement;
b) a non-weighted wearable equipment of the same type as the weighted wearable equipment, the non-weighted wearable equipment having a sensor and not including a weight enhancement; and
c) a training application functional communication with each of the weighted wearable equipment and the non-weighted wearable equipment and having a data processor that includes instructions for:
analyzing data received from the sensor of each of the weighted wearable equipment and the non-weighted wearable equipment and
generating predictive information derived from exercise training data from the sensors.

2. The training system of claim 1, wherein the type is gloves.

3. The training system of claim 1, wherein the sensors are selected from the group of sensors consisting of: accelerometers, gyroscopes, photoelectric sensors, position sensors, tilt sensors, pressure sensors, temperature sensors, blood pressure sensors, heart rate monitors, and SpO2 sensors.

4. The training system of claim 1, wherein the weight enhancement includes a plurality of weight bodies disposed in closed pockets within the weighted wearable equipment.

5. The training system of claim 1, wherein the instructions of the data processor include instructions for generating predictive information about ho ser will currently perform using the non-weighted wearable equipment based on generating a mapping rule by comparing historical data for that user from both the weighted wearable equipment sensor and the non-weighted wearable equipment sensor and by applying the mapping rule to current sensor data from the weighted wearable equipment sensor.

6. The training system of claim 1, wherein the type is selected from the group of types consisting of: gloves, shoes, belts, shoulder-pads, knee-pads, elbow-pads, helmets, wristbands, and shin-guards.

7. The training system of claim 1, wherein the data processor receives motion information from the sensors.

8. The training system of claim 1, further comprising an analysis module that includes the data processor, a data storage module functionally coupled to the analysis module such that the analysis module may call data therefrom, and a user interface module functionally coupled to the data processor module such that analysis therefrom may be reported to the user interface module on demand from a user.

9. A training system, comprising:
a) a weighted glove having an accelerometer and including a weight enhancement;
b) a non-weighted glove having an accelerometer and not including a weight enhancement; and
c) a training application in functional communication with each of the weighted glove and the non-weighted glove and having a data processor that includes instructions for:
analyzing exercise training data received from the accelerometer of each of the weighted wearable equipment and the non-weighted wearable equipment and
generating predictive performance data derived from analyzing the exercise training data.

10. The training system of claim 9, wherein the instructions for generating predictive performance data include instructions for generating a mapping rule by comparing historical data for that user from both the weighted glove and the non-weighted glove and by applying the mapping rule to current accelerometer data from the weighted glove.

11. The training system of claim 10, further comprising a user interface module disposed on a portable computing device in functional communication with the data processor such that a user of the portable computing device can receive predictive performance data therefrom.

12. The training system of claim 11, wherein the user interface module includes a user interface for an athlete account that is different from a user interface for a coach account and wherein each of the athlete account and the coach account can access the same set of training and predictive data over different mobile computing devices.

13. The training system of claim 12, wherein each of the weighted and non-weighted gloves includes a wireless communication module that transmits training data to a mobile computing device.

14. The training system of claim 13, wherein each of the weighted glove and non-weighted glove includes a plurality of sensor types.

15. A training system for use in weight-enhanced training techniques, comprising:
a first sensor module disposed within a first apparel;
a second sensor module disposed within a second apparel, the second apparel being of a same type as the first apparel but having a weight differential with respect to the first apparel;
an analysis module in functional communication with each of the first sensor module and the second sensor module, the analysis module including instructions for receiving and processing sensor information from each of the first sensor module and the second sensor module and associating such data with each respectively and wherein the analysis module includes information about the weight differential and utilizes that information in processing the sensor information.

16. The training system of claim 15, wherein each of the first apparel and second apparel are gloves that each include an accelerometer within the associated first and second sensor modules.

17. The training system of claim 15, further comprising a predictive module functionally coupled to the analysis module and including instructions for predicting performance of a user based on historical sensor data.

18. A training kit, comprising:
a) a weighted wearable equipment of a type having a sensor and including a weight enhancement;
b) a non-weighted wearable equipment of the same type as the weighted wearable sensor, the non-weighted wearable equipment having a sensor and not including a weight enhancement; and
c) instructions for accessing A training application that can analyze data received from the sensor of each of the weighted wearable equipment and the non-weighted wearable equipment and generate predictive information derived from exercise training data from the sensors.

19. The kit of claim 18, wherein the type is a glove and the sensor of each glove is an accelerometer.

20. A method of training, comprising the steps of:
collecting weighted training data for a user from a weighted wearable equipment of a type having a sensor and including a weight enhancement;
collecting non-weighted training data for the user from a non-weighted wearable equipment of the same type as the weighted wearable sensor, the non-weighted wearable equipment having a sensor and not including a weight enhancement;
analyzing weighted and non-weighted training data for the user in combination with information about a weight differential between the weighted wearable equipment and the non-weighted wearable equipment; and
generating predictive performance data for the user derived from analyzing the weighted and non-weighted training data.
